(19) United States
(12) Patent Application Publication
(10) Pub. No.: US 2009/0204012 A1
(43) Pub. Date: Aug. 13, 2009

(54) Apparatus and method for determining a physiological parameter

(75) Inventor: Stephan Joeken, Schopfheim (DE)

Correspondence Address: COLLARD & ROE, P.C., 1077 NORTHERN BOULEVARD, ROSLYN, NY, 11576, US

(73) Assignee: Pulsion Medical Systems AG

(21) Appl. No.: 12/322,652

(22) Filed: Feb. 5, 2009

(30) Foreign Application Priority Data
Feb. 7, 2008 (EP) 08101369.0

(51) Int. Cl. A61B 5/02

(52) U.S. Cl. 600/504

(57) ABSTRACT

An apparatus for determining a physiological parameter of a patient, such as cardiac output, comprises a sensor device providing readings of a blood variable, memory means storing the readings as a curve over time, evaluation means determining a mean value from the curve and determining the physiological parameter using the mean value is provided. The evaluation means determine a spectral density of the curve and/or a variance. The evaluation means also determine at least one model parameter representing an effective value of a heart beat using the mean value and the spectral density and/or variance. The effective value is selected from an effective amplitude of the heart beat, an effective duration of the heart beat and an effective area under the heart beat. The physiological parameter is determined using at least one of the model parameters.

The present invention relates to an apparatus for determining at least one physiological parameter of a patient. In particular, the invention relates to an apparatus for determining at least one physiological parameter of a patient, which comprises at least one sensor device adapted to provide readings of a blood variable of the patient, storage means for storing said readings as at least one curve representing said blood variable over time and evaluation means adapted to determine a mean value from said curve and to determine said at least one physiological parameter using said mean value.

In addition, the invention relates to a method for determining at least one physiological parameter of a patient by providing readings of a blood variable of the patient, storing said readings as at least one curve representing said blood variable over time, determining a mean value from said pressure curve and determining said at least one physiological parameter using said mean value.

Furthermore, the invention relates to a physical storage medium having stored thereon a computer program which, when executed on a computer system, causes the computer system to perform a method for determining at least one physiological parameter of a patient.

Devices and methods for determining cardiac function parameters such as cardiac output, ejection fractions or stroke volume are well known in the state of the art. Specifically, cardiac output is obtainable from continuous blood pressure waveforms, which are derived by conventional means via an arterial line measuring the arterial blood pressure. Generally, the cardiac output is equal to the stroke volume multiplied by the heart rate.

For example, U.S. Pat. No. 6,071,244 describes a method for the measurement of cardiac output in a patient, which involves the continuous measurement of the arterial blood pressure waveform, then running an autocorrelation of the waveform data and finally establishing the cardiac output by multiplying the transformed autocorrelation data with a calibration factor using a known accurate calibration method for that patient.

EP-A-0 947 941 describes in vivo determination of the compliance function and the cardiac output of a patient using pulse contour analysis.

However, the current reliable methods for continuously determining cardiac output and/or stroke volume require calibration to a reference value, which usually is determined by an additional discontinuous measurement. In addition, pulse contour analyses require the reliable detection of the dicrotic notch to separate the systolic from the diastolic pulse pressure components. Furthermore, in some circumstances, such as during major surgery, the waveform may not display a dicrotic notch at all. In addition, the above approaches are compromised by disturbances in the pressure measurement due to arrhythmia or reflections from the arterial branches.

Further, there is a general desire to reduce the degree of invasiveness in hemodynamic measurement procedures.

Therefore, it is an object of the present invention to provide an apparatus or method, respectively, of the type described above for measuring a physiological parameter such as cardiac output, ejection fractions or stroke volume, which negotiates the necessity for the calibration of the measurement data with a reference value obtained by an additional discontinuous measurement for the patient.

A further object of the present invention is to provide an apparatus or method, respectively, of the type described above, which does away with the requirement to detect the dicrotic notch.

Additionally, it is a particular object of the current invention to minimize the said measurement disturbances in an apparatus or method as described above due to arrhythmia or reflections from the arterial branches.

It is further an object to at least not increase the degree of invasiveness involved in determining the physiological parameter(s).

According to one aspect of the present invention, these objects are achieved by an apparatus of the type initially mentioned, wherein the evaluation means of the apparatus are further adapted
to determine at least one of a spectral density S(o) of the curve and a variance of the arterial blood variable,
to determine at least one model parameter representing an effective value of a heart beat using the determined mean value and at least one of said spectral density S(ω) and said variance, said effective value being selected from an effective amplitude A_{eff} of said heart beat, an effective duration d_{eff} of said heart beat and an effective area F_{eff} under said heart beat, and
   to determine the at least one physiological parameter using at least one said model parameter.

In a preferred embodiment, these objects are achieved by an apparatus for determining at least one physiological parameter of a patient, said apparatus comprising:
a pressure sensor device adapted to provide readings of an arterial blood pressure of said patient,
memory means for storing said readings as a pressure curve P(t) over time t,
evaluation means adapted to determine a mean arterial blood pressure <P> from said pressure curve P(t) and to determine said at least one physiological parameter using said mean arterial blood pressure <P>,
   wherein said evaluation means are further adapted
to determine at least one of a spectral density S(ω) of said pressure curve and a variance <(δP)²> of said arterial blood pressure,
to determine at least one model parameter representing an effective value of a heart beat using said mean arterial blood pressure <P> and at least one of said spectral density S(ω) and said variance <(δP)²>, said effective value being selected from an effective amplitude A_{eff} of said heart beat, an effective duration d_{eff} of said heart beat and an effective area F_{eff} under said heart beat, and
to determine said at least one physiological parameter using at least one said model parameter.

Though the present invention will be described focusing on this preferred embodiment hereinafter, the present invention can also be carried out in an equally advantageous manner using sensor setups other than a pressure sensor device, yet following the same or widely analogous evaluation principle. For example, the present invention can be advantageously carried out using an (opto)plethysmography and/or pulse-oximetry sensor setup and basing the evaluation techniques on the plethysmography and/or pulse-oximetry readings, which may usually include a plurality of intensity signals.

The evaluation techniques described hereinafter on the basis of blood pressure readings may thus advantageously be implemented in an analogous manner using a curve representing a local blood volume, local blood perfusion variable or the like instead of the pressure curve, and a mean value of such a variable instead of the mean arterial blood pressure. I.e., in the evaluation techniques described hereinafter, the blood pressure dependent terms may be substituted by terms depending on another variable indicative of the perfusion condition of an artery.

It is to be understood that the curve may directly be derived from raw data such as an electrical signal depending on a local light transmission and/or reflection detected by an optical sensor arrangement.

As (opto-)plethysmography and/or pulse-oximetry sensor setups can easily be implemented using non-invasive sensors (e.g. finger clip sensors, ear clip sensors or sensors affixed to the forehead), the present invention thus allows to greatly decrease or eliminate the invasiveness of physiological, in particular hemodynamic, parameter determination.

(Opto-)plethysmography and pulse-oximetry sensors per se are widely known from the prior art. Plethysmography is a technique for measuring blood volume variations correlating with a subject's pulse. This technique allows implementation of continuous non-invasive blood pressure or pulse frequency measurements, respectively. Pulse-oximetry measurements allow determining oxygen saturation of the blood. In optoplethysmography, there are mainly two alternative measurement principles: transmission measurements and reflexion measurements. Transmission measurements are based on measurement of intensity according to Lambert-Beer Law. In optoplethysmography usually two or more wavelengths are used within the range of approximately 600 nm through approximately 1000 nm, usually about 660 nm and 940 nm. Suitable measurement sites are, e.g., fingertips, ear lobes, forehead (for reflexion measurements), nose and toes. Technical sensor implementations have been widely published both in patent and non-patent literature, such as Vincent Chan, Steve Underwood: “A Single-Chip Pulsoximeter Design Using the MSP430” SLAA274—November 2005 (http://focus.ti.com/lit/an/slaa274/slaa274.pdf) and Texas Instruments Medical Applications Guide 1Q2009, p. 37-44 (http://focus.ti.com/lit/an/slyb108d/slyb108d.pdf).

Preferably, said at least one physiological parameter includes at least one of stroke volume SV, cardiac output CO and ejection fraction EF.

It is further preferred that said evaluation means are adapted to determine said stroke volume SV as a product of a first model parameter representing said effective amplitude A_{eff} and a second model parameter representing said effective duration d_{eff}.

In addition, it is preferred that said evaluation means are adapted to determine said ejection fraction EF as a product of a model parameter representing said effective duration d_{eff} and a heart rate HR of said patient.

In particular preferred is that said evaluation means are adapted to determine said cardiac output CO as a product of a first model parameter representing said effective amplitude A_{eff}, a second model parameter representing said effective duration d_{eff} and an approximation of a heart rate of said patient.

Preferably, said approximation of said heart rate is selected from an actual measured heart rate HR and an approximate function λ_{eff}, said approximate function λ_{eff} including a quotient with a dividend comprising the square of the mean arterial pressure <P> and a divisor comprising the spectral density S(ω) at ω=0.

Further preferably, said evaluation means are adapted to use a correction parameter α in determining said model parameter, said correction parameter α assuming values greater than or equal to 1, said values being the higher, the less the patient's heart frequency deviates from a rhythmic condition.

In addition, it is preferred that said evaluation means are adapted to use a monotonous correction function σ depending on said correction parameter α and assuming values from 0 to 1, wherein said correction function σ assumes the value of 0 if said correction parameter α equals 1 and said correction function σ assumes the value of 1 for said correction parameter α tending to infinity.

Preferably, said effective amplitude A_{eff} is provided as a quotient with a dividend comprising the sum of said variance <(δP)²> and the product of said correction function σ and the square of the mean arterial pressure <P> and a divisor comprising the mean arterial pressure <P>.

Furthermore preferably, said effective amplitude d_{eff} is provided as a quotient with a dividend comprising the spectral density S(ω) at ω=0 and a divisor comprising the sum of said variance <(δP)2> and the product of said correction function σ and the square of the mean arterial pressure <P>.

Also preferably, said apparatus provides Fourier Transformation means for determining said spectral density S(ω) as the Fourier Transformation of the autocorrelation of said pressure curve.

Preferably, the spectral density S(ω) at ω=0 is provided as said variance <(δP)²> multiplied by a constant factor.

In a preferred embodiment, said evaluation unit is further adapted to determine a comparative value of at least one of said physiological parameters from said pressure curve using pulse contour algorithms.

In an additional preferable embodiment, said apparatus further comprises
means for administering a travelling deviation of an intrinsic physical quantity (such as temperature, an optical/spectral property or an indicator concentration) to the blood stream of said patient at a first location of the blood circulation of said patient, and
sensor means for measuring said physical quantity at a second location of the blood circulation of said patient over the course of time,
   wherein, said memory means are adapted to record said physical quantity measured over the course of time at said second location as a dilution curve, and said evaluation unit is adapted to determine a comparative value of at least one of said physiological parameters from said dilution curve using dilution algorithms.

Preferably, said evaluation unit is adapted to use said comparative value for calibration.

Preferably, said calibration includes determining, using said comparative value, a correction parameter α used in determining said model parameter, said correction parameter α assuming values greater than or equal to 1, said values being the higher the less the patient's heart frequency deviates from a rhythmic condition.

In a preferred embodiment, said evaluation unit is adapted to reject and re-calculate said physiological parameter, if the difference between the determined physiological parameter and the respective comparative value exceeds a threshold value.

Further to these embodiments, the above objects are achieved under an additional aspect of the invention by a method for determining at least one physiological parameter of a patient, said method comprising the steps of:
importing readings of a blood variable of said patient,
storing said readings as a curve representing said blood variable over time t,
determining a mean value from said curve and determining at least one physiological parameter using said mean value
   wherein the method further includes
determining at least one of a spectral density S(ω) of said curve and a variance of said blood variable,
determining at least one model parameter representing an effective value of a heart beat using said mean value and at least one of said spectral density S(ω) and said variance, said effective value being selected from an effective amplitude A_{eff} of said heart beat, an effective duration d_{eff} of said heart beat and an effective area F_{eff} under said heart beat, and
determining said at least one physiological parameter using at least one said model parameter.

In a preferred embodiment, the above objects are achieved by a method for determining at least one physiological parameter of a patient, said method comprising the steps of:
importing readings of an arterial blood pressure of said patient,
storing said readings as a pressure curve P(t) over time t,
determining a mean arterial blood pressure <P> from said pressure curve P(t) and determining at least one physiological parameter using said mean arterial blood pressure <P>,
   wherein the method further includes
determining at least one of a spectral density S(ω) of said pressure curve and a variance <(δP)²> of said arterial blood pressure,
determining at least one model parameter representing an effective value of a heart beat using said mean arterial blood pressure <P> and at least one of said spectral density S(•) and said variance <(δP)²>, said effective value being selected from an effective amplitude A_{eff} of said heart beat, an effective duration d_{eff} of said heart beat and an effective area F_{eff} under said heart beat, and
determining said at least one physiological parameter using at least one said model parameter.

Preferably, in above method said at least one physiological parameter includes at least one of stroke volume SV, cardiac output CO and ejection fraction EF.

As mentioned in connection with the above apparatus, the method described hereinafter on the basis of blood pressure readings may advantageously be implemented in an analogous manner using a curve representing a local blood volume variable, local blood perfusion variable or the like instead of the pressure curve, and a mean value of such a variable instead of the mean arterial blood pressure. I.e., in the method described hereinafter, the blood pressure dependent terms may be substituted by terms depending on another variable indicative of the perfusion condition.

It can be particularly advantageous to carry out the inventive method using plethysmography and/or pulse-oximetry readings (which may usually include a plurality of intensity signals), because (opto-)plethysmography and/or pulse-oximetry measurements can easily be implemented in a non-invasive manner.

Furthermore preferably in above method, said stroke volume SV is determined as a product of a first model parameter representing said effective amplitude A_{eff} and a second model parameter representing said effective duration d_{eff}.

It is further preferred that in above method said ejection fraction EF is determined as a product of a model parameter representing said effective duration d_{eff} and a heart rate HR of said patient.

Further preferably, in above method said cardiac output CO is determined as a product of a first model parameter representing said effective amplitude A_{eff}, a second model parameter representing said effective duration d_{eff} and an approximation of a heart rate of said patient.

Also preferably in above method, said approximation of said heart rate is selected from an actual measured heart rate HR and an approximate function λ_{eff}, said approximate function λ_{eff} including a quotient with a dividend comprising the square of the mean arterial pressure <P> and a divisor comprising the spectral density S(ω) at ω=0.

In addition, in above method, it is preferred that a correction parameter α is used in determining said model parameter, said correction parameter α assuming values greater than or equal to 1, said values being the higher the less the patient's heart frequency deviates from a rhythmic condition.

Preferably, in above method, a monotonous correction function σ is used depending on said correction parameter α and assuming values from 0 to 1, wherein said correction function σ assumes the value of 0 if said correction parameter α equals 1 and said correction function σ assumes the value of 1 for said correction parameter α tending to infinity.

In a preferred embodiment of above method, said effective amplitude A_{eff} is provided as a quotient, the dividend of said quotient comprising the sum of said variance <(δP)²> and the product of said correction function σ and the square of the mean arterial pressure <P> and the divisor of said quotient comprising the mean arterial pressure <P>.

Preferably, in above method, said effective amplitude d_{eff} is provided as a quotient with a dividend comprising the spectral density S(ω) at ω=0 and a divisor comprising the sum of said variance <(δP)²> and the product of said correction function σ and the square of the mean arterial pressure <P>.

It is further preferred that in above method, said spectral density S(ω) is determined as the Fourier Transformation of the autocorrelation of said pressure curve.

Preferably, in above method, the spectral density S(ω) at ω=0 is provided as said variance <(δP)²> multiplied by a constant factor.

Preferably, in above method, a comparative value of at least one of said physiological parameters is determined from said pressure curve using pulse contour algorithms.

In a preferred embodiment, above method further comprises
inputting information about a traveling deviation of an intrinsic physical quantity (such as temperature, an optical/spectral property or an indicator concentration) administered to the blood stream of said patient at a first location of the blood circulation of said patient, and
reading in measurement readings of said physical quantity at a second location of the blood circulation of said patient over the course of time,
   wherein, said measurement readings of said physical quantity at said second location of the blood circulation of said patient are recorded as a dilution curve, and said comparative value of at least one of said physiological parameters is determined from said dilution curve using dilution algorithms.

Preferably, in above method, said comparative value is used for calibration.

In addition in above method, it is preferred that said calibration includes determining, using said comparative value, a correction parameter α used in determining said model parameter, said correction parameter a assuming values greater than or equal to 1, said values being the higher the less the patient's heart frequency deviates from a rhythmic condition.

Preferably, in above method, said physiological parameter is rejected and re-calculated, if the difference between the determined physiological parameter and the respective comparative value exceeds a threshold value.

An additional embodiment of the present invention comprises a physical storage medium having stored thereon a computer program which, when executed on a computer system, causes the computer system to perform a method as described above.

Generally, any of the embodiments described or options mentioned herein may be particularly advantageous depending on the actual conditions of application. Further, features of one embodiment may be combined with features of another embodiment as well as features known per se from the prior art as far as technically possible and unless indicated otherwise.

In the following, the invention is described in more detail. The accompanying drawings, which are schematic flow diagrams and graphs, serve for a better understanding of the features of the current invention.

Therein:

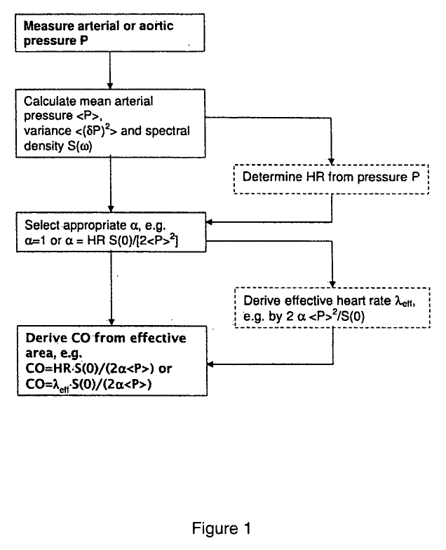

FIG. 1 is a flow diagram illustrating a preferred embodiment of the inventive method for determining the cardiac output CO. Optional features are presented in dashed lines. The quantity α may depend on the CVP (central venous pressure) and/or the heart rate variability,

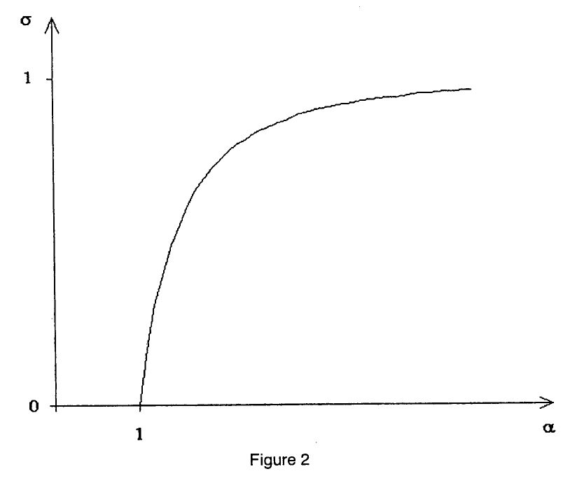

FIG. 2 is an exemplary plot of the functional relationship σ(α), i.e. the behaviour of σ for typical values of α, according to one embodiment of the present invention

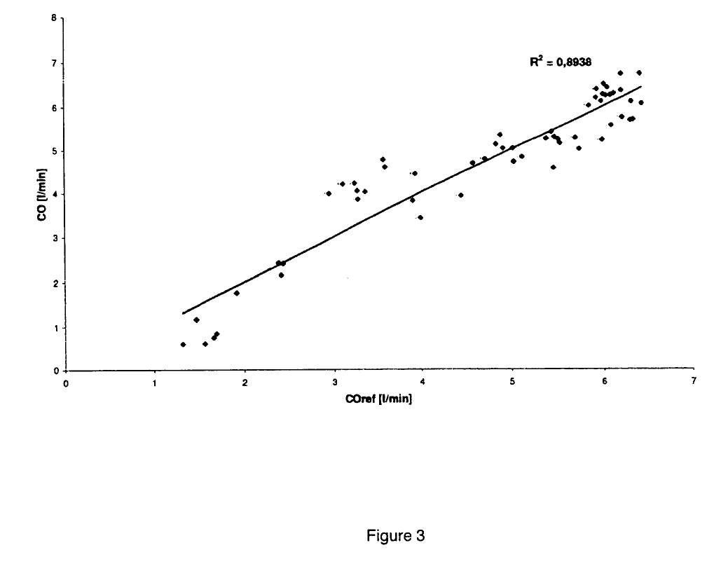

FIG. 3 shows a correlation plot between the CO values determined according to an embodiment of the present invention by eq. (24) and the reference values CO_{ref} obtained by the transpulmonary cardiac output measurements.

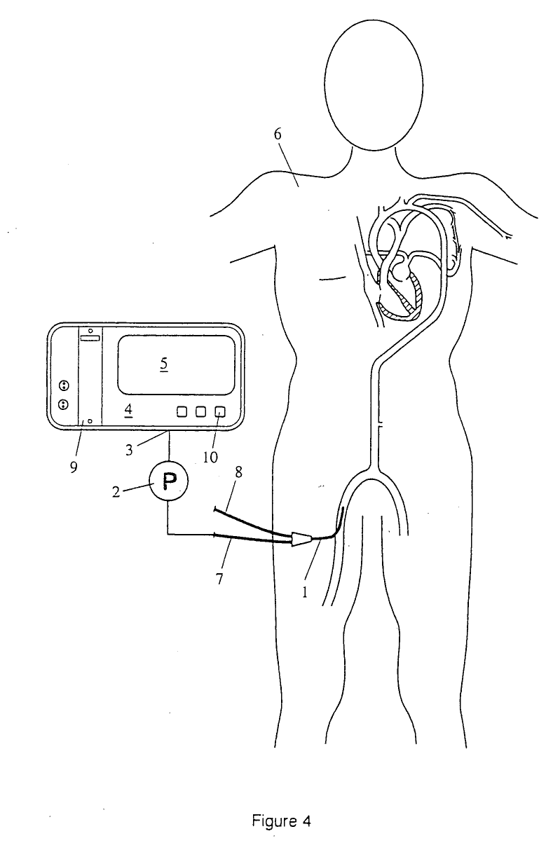

FIG. 4 illustrates the general setup of an apparatus according to an embodiment of the present invention making use of an arterial pressure sensor.

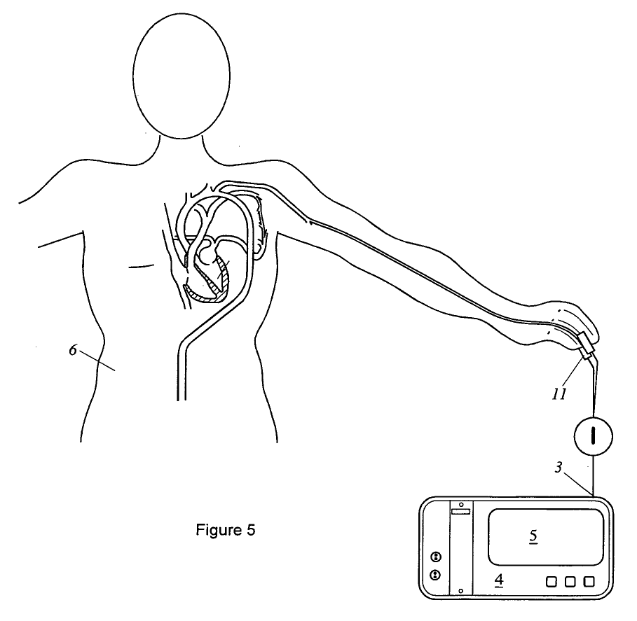

FIG. 5 illustrates the general setup of an apparatus according to a different embodiment of the present invention making use of an optoplethysmography sensor.

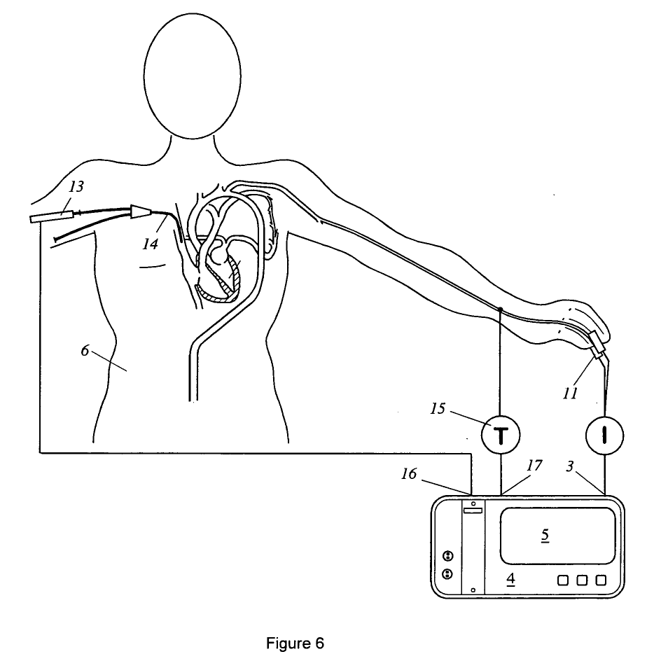

FIG. 6 shows a modified setup similar to FIG. 5.

FIG. 1 illustrates (indicated by regular lines) the most important steps of one preferred embodiment of the present invention. Dashed lines indicate optional steps for modifying the embodiment to yield another preferred embodiment. In order to improve the illustration of the preferred embodiments and to ease the understanding thereof, the following description also attempts to describe in more detail the underlying calculatory principles.

While the main focus of the described embodiments is on cardiac output determination, other physiological parameters may also be established with the apparatus and methods according to the invention, in particular, such parameters characterising cardiac function, such as ejection fractions or stroke volume.

As is shown in FIG. 1, the current invention makes use of measurement readings representing the arterial or aortic pressure P, the readings being provided either as raw data (such as voltage or electric current) from a suitable sensor or already in a pre-processed state. Performing the actual measurements may be achieved in many ways, such as employing a suitable arterial catheter assembly with a pressure transducer or applying non-invasive blood pressure measurements, and is well known from the prior art and not part of the inventive method itself. The measured pressure depends on time t and results from the superposition of several heart beats bₖ(t−tₖ), beginning at time tₖ. Due to reflections in the arterial tree, several heart beats may contribute to the resulting pressure, which can be described by the following function:

$$P ( t ) = {\sum }_{k} {b}_{k} ( t - {t}_{k} ) \qquad ( 1 )$$

In general, all beats may differ from each other, however, for a limited period of time, an average beat b(t) exists so that the function of equation (1) can be described adequately by

$$\begin{matrix}P ( t ) = {\sum }_{k} {b}_{k} ( t - {t}_{k} ) & ( 2 ) \\ \approx {\sum }_{k} b ( t - {t}_{k} ) & ( 3 )\end{matrix}$$

As in healthy living beings the heart rate HR usually does not vary strongly within a limited time frame (e.g. 10 s to 200 s), successive beats are almost equidistant and the occurrence of beats is determined by tₖ=k/HR.

During strong arrhythmia, however, heart beats occur very irregularly. The worst case scenario is the random occurrence of independent beats, which can be described by a Poisson-Process, i.e. the intervals between successive heart beats are distributed at random according to an exponential-distribution q such that:

q(tₖ−tₖ₋₁)=HR*exp(−HR*(tₖ−tₖ₋₁)).

In this case, the superposition of equation (3) is a Campbell-Process, as is described in: [N. R. Campbell, “The study of discontinuous phenomena.” Proc. Camp. Philos. Soc. Math. Phys. Sci. 15:117-136,1909.]

According to the present embodiment, the cardiac function can be assessed in both conditions, i.e. for regular and irregular heart activity, by means of the mean, variance and spectral density of the measured pressure function P(t),

The mean arterial pressure <P> is derived from the integral of the measured pressure P(t) over an appropriate time span T, i.e.

$$M A P = < P > = \frac{1}{T} {\int }_{0}^{T} P ( t ) d t \qquad ( 4 )$$

The Variance of the pressure is calculated from δP(t)=P(t)−<P> so that:

$$< {( δ P ( t ) )}^{2} > = \frac{1}{T} {\int }_{0}^{T} {( P ( t ) - < P > )}^{2} d t \qquad ( 5 )$$

The Spectral density is the Fourier Transformation of the autocorrelation of P(t) and hence is determined by:

$$S ( ω ) = 4 {\int }_{0}^{∞} cos ( ωτ ) < δ P ( t ) * δP ( t - τ ) > d τ \qquad ( 6 )$$

In the case of arrhythmia, the arrhythmic conditions can be described by a Campbell-Process application of equations (4) to (6), which results in the following functions:

$$\begin{matrix}< P > = H R * {\int }_{0}^{∞} b ( t ) d t & ( 7 ) \\ < {( δ P )}^{2} > = H R * {\int }_{0}^{∞} {b}^{2} ( t ) d t & ( 8 ) \\ S ( ω ) = 2 * H R * {\vert \tilde{b} ( ω ) \vert}^{2} & ( 9 )\end{matrix}$$

with the Fourier Transformation \tilde{b}(ω) of the average beat b(t):

$$\tilde{b} ( ω ) = {\int }_{0}^{∞} b ( t ) * {e}^{- iω t} d t \qquad ( 10 )$$

For rhythmic conditions with periodic heart beats, the application of equations (4) to (6) results to:

$$\begin{matrix}< P > = H R * {\int }_{0}^{∞} b ( t ) d t & ( 11 ) \\ < {( δ P )}^{2} > = H R * {\int }_{0}^{∞} {b}^{2} ( t ) d t - < P {>}^{2} & ( 12 ) \\ S ( ω ) = 2 α * H R * {\vert \tilde{b} ( ω ) \vert}^{2} , with α > 1 & ( 13 )\end{matrix}$$

The parameter α depends on the heart rate variability on a time scale much larger than T. For practical implementations, α can be determined through imperical studies or simply estimated taking into account the principles described herein.

Merging the different conditions can be based on the following considerations. Equations (7) to (9) and (11) to (13) have the same structure. In particular, α in eq. (13) decreases with a decreasing accuracy in the periodicity of the beats b(t). Finally, for randomly occurring beats, α tends to the lower limit α=1 and the equations (13) and (11) become identical. Equations (12) and (8) can also be merged. Therefore, A function σ(α) is introduced, which tends to 0 as α tends to 1, and σ(α) tends to 1 for α>>1. This relationship is described in an exemplary manner in FIG. 2.

In consequence, the rhythmic, arrhythmic and intermediate activity of the heart is described by:

$$\begin{matrix}< P > = H R * {\int }_{0}^{∞} b ( t ) d t & ( 14 ) \\ < {( δ P )}^{2} > = H R * {\int }_{0}^{∞} {b}^{2} ( t ) d t - σ ( α ) < P {>}^{2} & ( 15 ) \\ S ( ω ) = 2 α * H R * {\vert \tilde{b} ( ω ) \vert}^{2} & ( 16 )\end{matrix}$$

wherein α and σ are limited as follows:

| | α | σ (alpha) |
|---|---|---|
| arrhythmic | 1 | 0 |
| intermediate | ≧1 | >0 and <1 |
| rhythmic | >1 | 1 |

Furthermore, the acquired data may be interpreted so as to afford a characterisation of the average beat in terms of its amplitude A and duration d.

$$\begin{matrix}A := \frac{\int {b}^{2} ( t ) d t}{\int b ( t ) d t} & ( 17 ) \\ d := \frac{{[ \int b ( t ) d t ]}^{2}}{\int {b}^{2} ( t ) d t} & ( 18 )\end{matrix}$$

Thus, if a square wave is assumed,

b(t)=A, for 0≦t≦d

b(t)=0, otherwise (19)

Consequently, introducing the equations (14) to (16) yields:

$$\begin{matrix}{A}_{eff} = \frac{< {( δ P )}^{2} > + σ < P {>}^{2}}{< P >} & ( 20 ) \\ {d}_{eff} = \frac{S ( 0 )}{2 α * ( < {( δ P )}^{2} > + σ < P {>}^{2} )} & ( 21 )\end{matrix}$$

Therein, the index “eff” was introduced to point out that A_{eff} and d_{eff} are effective values reconstructed from the measured pressure signal P(t). It is emphasized that this reconstruction is possible in all conditions, even if no heart beats are detectable in the pressure curve P(t). Moreover, the product of the area A_{eff} and duration d_{eff} results in the area under the effective beat F_{eff}, which is

$$\begin{matrix}{F}_{eff} = {A}_{eff} * {d}_{eff} \\ = \frac{S ( 0 )}{2 α < P >}\end{matrix} \qquad ( 22 )$$

even if the beats do not represent a square wave.

In addition, the ratio

$${λ}_{eff} = \frac{2 α < P {>}^{2}}{S ( 0 )} \qquad ( 23 )$$

gives an approximation of the heart rate in all circumstances.

According to the present embodiments, the obtained parameters are used to characterize a patient's cardiac function. In particular, the quantities A_{eff}, d_{eff}, λ_{eff} and combinations thereof are useful to characterize stroke volume SV, cardiac output CO, ejection fraction EF and others.

Specifically, the Cardiac output CO may be derived according to

$$\begin{matrix}C O = H R * {A}_{eff} * {d}_{eff} \\ = H R * \frac{S ( 0 )}{2 α < P >}\end{matrix} \qquad ( 24 )$$

In order to test the values obtained with the inventive method, a comparison with a known standard reference method was undertaken. For this purpose the cardiac output was measured according to the present invention and also with the established thermodilution technique according to U.S. Pat. No. 5,526,817.

The results of this comparison are displayed in FIG. 3. Therein, the abscissa represents cardiac output in liters per minute as determined by the reference method and the ordinate represents output in liters per minute as determined by the inventive method. Accordingly, the results achieved for the cardiac output in both measurements are in good agreement.

Furthermore, the other cardiac functions stroke volume and ejection fraction are also readily available from the parameters determined according to the inventive method.

The stroke volume SV is afforded by dividing the cardiac output by the heart rate HR; thus: SV=A_{eff}*d_{eff}.

Further, the quantity d_{eff}*HR corresponds to the ejection fraction EF. Depending on the location of the pressure measurement (pulmonary artery or systemic artery) the readings of which are used, this might by either the left or the right ventricular ejection fraction.

Further embodiments of the invention will be described below.

An optional simplification to the inventive method may be made, if the spectral density is required for frequencies f close to or at ω=2πf=0. Then according to eq. (6), the variance of the pressure P can be used to approximate

$$\begin{matrix}S ( 0 ) \approx 2 * ( < {P}^{2} > - < P {>}^{2} ) & ( 25 ) \\ = 2 * < {( δ P )}^{2} > & ( 26 )\end{matrix}$$

This simplification can also be used in the other relations, particularly, in eq. (24).

In addition, an arrhythmia adjustment may be made since the coefficient 1/α may depend on the heart rate variability.

Furthermore, the central venous pressure CVP may influence the coefficient 1/α. Therefore, in order to establish α, CVP can be taken into account either by reading in measurement data directly as provided by a CVP measurement source or by user input. Generally, measuring CVP per se is well known from the prior art and not part of the inventive method.

The effect of a varying heart rate on the current invention can be detected by determining the number of heart beats within a pressure sample and the effective heart rate λ_{eff} determined by equation (23). Particularly, the ratio of HR and λ_{eff} leads to

$$α = \frac{H R * S ( 0 )}{2 < P {>}^{2}} \qquad ( 27 )$$

Therefore, an adjustment of a patient monitoring system implementing the present invention to the current heart rate variability is possible.

The Spectral density S(0) for ω=2πf=0 can also be determined differently, e.g.
a) by means of the spectral density for frequencies tending to zero, i.e. S(0)=lim_{ω→o}S(ω)=lim_{f→o}S(2πf), or
b) by fitting an appropriate function (e.g. S(ω)=S(0)/[1+(ωτ)²]ⁿ⁺¹) to the measured spectrum. Therein, local maxima corresponding to the heart rate should preferably be neglected.

FIG. 4 shows the general setup of an apparatus according to an embodiment of the present invention. An arterial catheter 1 is equipped with a pressure sensor for measuring arterial blood pressure. The pressure sensor of the catheter 1 is connected, via a pressure transducer 2, to an input channel 3 of a patient monitoring apparatus 4. Beside a proximal port 7 used to acquire the pressure signal, the catheter 1 may comprise one or more other proximal ports 8 to perform additional functions, such as blood temperature measurements or the like. The patient monitoring apparatus 4 contains appropriate storage means for storing the readings of blood pressure over time and serving as a processing storage.

The patient monitoring also comprises a computing facility 9, which may include a digital signal processing instance 9, which is programmed to perform calculations in accordance with the equations described above, a display 5 to visualize the determined parameters (as numeric values, graphically or both) and control elements 10 to operate said apparatus. In addition, the determined parameters may be stored at a recording medium and/or printed. For this purpose, the patient monitoring apparatus 4 may comprise various interface ports for connecting peripheral equipment.

A particularly preferred embodiment requires a single arterial pressure sensor only. Though the sensor is shown to be invasive using said catheter 1, a non-invasive pressure sensor may be implemented instead. Instead of (or in addition to) an arterial catheter 1 as shown, a pulmonary artery catheter may also be used, in particular, if right ventricular ejection fraction is to be determined.

Various implementations of the invasive pressure sensor assembly can be particularly advantageous. Pressure can either be transmitted hydraulically to a proximal catheter port 7 and measured by an external sensor or may be measured directly on-site using a sensor installed at or near the catheter tip. Capacitative sensors, piezo sonsors or optical pressure sensors (e.g. based on Fabry-Perot interferometry) may be used.

With reference to the procedure described in FIG. 1, a first pressure signal P is measured by said apparatus depicted in FIG. 4, e.g. from arteria femoralis, arteria radialis, arteria brachialis or arteria pulmonalis or any other appropriate arterial vessel downstream of the left or right ventricle, respectively.

By means of equation (6), the computing facility 9 calculates the mean arterial pressure, the variance and the spectral density for the pressure measured within an appropriate time frame (e.g. 10 s to 200 s). For calculating the spectral density, designated fast fourier transformation means may be employed.

Furthermore, for ω=0 and α=1 the computing facility 9 employs equation (22) to calculate the effective area F_{eff}. The relationship between the effective area F_{eff} and the stroke volume SV is given by 1/α, wherein the latter can be determined empirically or alternatively, with respect to reference SV measurements (e.g. transpulmonary thermodilution) from the treated patient. In order to perform such reference SV measurements, the patient monitoring apparatus 4 may be equipped accordingly, e.g. by providing additional input channels for thermodilution measurement readings and by implementing suitable thermodilution algorithms in the computing facility 9, as known per se from the prior art.

The knowledge of SV permits the determination of CO according to CO=HR*SV, wherein the heart rate HR can also, optionally, be determined from the pressure and the spectral density or by other methods.

Although the embodiments described hereinabove in detail are based on an arterial pressure measurement, the invention may be carried out in an analogous manner on plethysmographic measurements, as mentioned in the introductory part of this description. FIG. 5 shows a setup with an optoplethysmographic finger-clip sensor 11 employing a photometric means for measuring transmitted light intensity and transmitting a respective signal to the input channel 3 of the patient monitoring apparatus 4. The patient monitoring apparatus 4 contains appropriate storage means for storing the photometer readings over time and serving as a processing storage.

The patient monitoring also comprises a computing facility 9, which may include a digital signal processing instance 9, which is programmed to perform calculations in accordance with equations as described above, wherein, however, the pressure curve and terms derived therefrom are substituted by respective terms based on the plethysmography measurement.

As in the other embodiments above, a display 5 to visualize the determined parameters (as numeric values, graphically or both) and control elements 10 to operate said apparatus are also provided. In addition, the determined parameters may be stored at a recording medium and/or printed. For this purpose, the patient monitoring apparatus 4 may comprise various interface ports for connecting peripheral equipment.

The embodiment depicted in FIG. 6 is generally set up as the embodiment of FIG. 5 but additionally comprises an injection channel 13 for administering a bolus of a temperature below blood temperature through a central venous catheter 14 to the blood stream in the vena cava sperior of the patient, and a temperature sensor 15 for measuring an arterial blood temperature over the course of time. Both the arterial blood temperature and a signal indicating temperature of the injected bolus and the time of injection are provided to the patient monitoring apparatus 4 via additional input channels 16 and 17, respectively. The memory means of the patient monitoring apparatus 4 record the arterial blood temperature measured over the course of time to determine a thermodilution curve therefrom. The patient monitoring apparatus 4 is adapted to determine, using thermodilution algorithms known per se, at least one comparative value of a physiological parameter from the dilution curve in order to allow calibration of the system.

1. Apparatus for determining at least one physiological parameter of a patient, said apparatus comprising:
a sensor device adapted to provide readings of an blood variable of said patient,
memory means for storing said readings as a curve representing said variable over time t,
evaluation means adapted to determine a mean value from said curve and to determine said at least one physiological parameter using said mean value,
wherein
said evaluation means are further adapted
to determine at least one of a spectral density S(ω) of said curve and a variance of said blood variable,
to determine at least one model parameter representing an effective value of a heart beat using said mean value and at least one of said spectral density S(ω) and said variance, said effective value being selected from an effective amplitude A_{eff} of said heart beat, an effective duration d_{eff} of said heart beat and an effective area F_{eff} under said heart beat, and
to determine said at least one physiological parameter using at least one said model parameter.

2. Apparatus according to claim 1, wherein said at least one physiological parameter includes at least one of stroke volume SV, cardiac output CO and ejection fraction EF.

3. Apparatus according to claim 2, wherein said evaluation means are adapted to determine said stroke volume SV as a product of a first model parameter representing said effective amplitude A_{eff} and a second model parameter representing said effective duration d_{eff}.

4. Apparatus according to claim 2, wherein said evaluation means are adapted to determine said ejection fraction EF as a product of a model parameter representing said effective duration d_{eff} and a heart rate HR of said patient.

5. Apparatus according to claim 2, wherein said evaluation means are adapted to determine said cardiac output CO as a product of a first model parameter representing said effective amplitude A_{eff}, a second model parameter representing said effective duration d_{eff} and an approximation of a heart rate of said patient.

6. Apparatus according to claim 5, wherein said approximation of said heart rate is selected from an actual measured heart rate HR and an approximate function λ_{eff}, said approximate function λ_{eff} including a quotient with a dividend comprising the square of the mean value and a divisor comprising the spectral density S(ω) at ω=0.

7. Apparatus according to claim 1, wherein said evaluation means are adapted to use a correction parameter α in determining said model parameter, said correction parameter α assuming values greater than or equal to 1, said values being the higher, the less the patient's heart frequency deviates from a rhythmic condition.

8. Apparatus according to claim 7, wherein said evaluation means are adapted to use a monotonous correction function a depending on said correction parameter α and assuming values from 0 to 1, wherein said correction function σ assumes the value of 0 if said correction parameter α equals 1 and said correction function σ assumes the value of 1 for said correction parameter α tending to infinity.

9. Apparatus according to claim 8, wherein said effective amplitude A_{eff} is provided as a quotient with a dividend comprising the sum of said variance and the product of said correction function σ and the square of the mean value and a divisor comprising the mean value.

10. Apparatus according to claim 8, wherein said effective amplitude d_{eff} is provided as a quotient with a dividend comprising the spectral density S(ω) at ω=0 and a divisor comprising the sum of said variance and the product of said correction function σ and the square of the mean value.

11. Apparatus according to claim 1 comprising Fourier Transformation means for determining said spectral density S(ω) as the Fourier Transformation of the autocorrelation of said curve.

12. Apparatus according to claim 1, wherein the spectral density S(ω) at ω=0 is provided as said variance multiplied by a constant factor.

13. Apparatus according to claim 1, wherein said evaluation unit is further adapted to determine a comparative value of at least one of said physiological parameters using pulse contour algorithms.

14. Apparatus according to claim 1, further comprising
means for administering a traveling deviation of an intrinsic physical quantity to the blood stream of said patient at a first location of the blood circulation of said patient, and
sensor means for measuring said physical quantity at a second location of the blood circulation of said patient over the course of time,
wherein said memory means are adapted to record said physical quantity measured over the course of time at said second location as a dilution curve, and said evaluation unit is adapted to determine a comparative value of at least one of said physiological parameters from said dilution curve using dilution algorithms.

15. Apparatus according to claim 13, wherein said evaluation unit is adapted to use said comparative value for calibration.

16. Apparatus according to claim 15, wherein calibration includes determining, using said comparative value, correction parameter α used in determining said model parameter, said correction parameter α assuming values greater than or equal to 1, said values being the higher the less the patient's heart frequency deviates from a rhythmic condition.

17. Apparatus according to claim 13, wherein said evaluation unit is adapted to reject and re-calculate said physiological parameter, if the difference between the determined physiological parameter and the respective comparative value exceeds a threshold value.

18. Apparatus according to claim 1, wherein said sensor device includes a pressure sensor device, said blood variable is an arterial blood pressure, said curve representing said variable over time is a pressure curve P(t) over time t, and said mean value is a mean arterial blood pressure <P>.

19. Apparatus according to claim 1, wherein said sensor device includes a plethysmography and/or pulse-oximetry sensor setup.

20. Method for determining at least one physiological parameter of a patient, said method comprising the steps of:
importing readings of a blood variable of said patient,
storing said readings as a curve representing said blood variable over time t,
determining a mean value from said curve and determining at least one physiological parameter using said mean value
wherein
said method further includes
determining at least one of a spectral density S(ω) of said curve and a variance of said blood variable,
determining at least one model parameter representing an effective value of a heart beat using said mean value and at least one of said spectral density S(ω) and said variance, said effective value being selected from an effective amplitude A_{eff} of said heart beat, an effective duration d_{eff} of said heart beat and an effective area F_{eff} under said heart beat, and
determining said at least one physiological parameter using at least one said model parameter.

21. Method according to claim 20, wherein said at least one physiological parameter includes at least one of stroke volume SV, cardiac output CO and ejection fraction EF.

22. Method according to claim 21, wherein said stroke volume SV is determined as a product of a first model parameter representing said effective amplitude A_{eff} and a second model parameter representing said effective duration d_{eff}.

23. Method according to claim 21, wherein said ejection fraction EF is determined as a product of a model parameter representing said effective duration d_{eff} and a heart rate HR of said patient.

24. Method according to claim 21, wherein said cardiac output CO is determined as a product of a first model parameter representing said effective amplitude A_{eff}, a second model parameter representing said effective duration d_{eff} and an approximation of a heart rate of said patient.

25. Method according to claim 24, wherein said approximation of said heart rate is selected from an actual measured heart rate HR and an approximate function λ_{eff}, said approximate function λ_{eff} including a quotient with a dividend comprising the square of the mean value and a divisor comprising the spectral density S(ω) at ω=0.

26. Method according to claim 20, wherein a correction parameter α is used in determining said model parameter, said correction parameter α assuming values greater than or equal to 1, said values being the higher the less the patient's heart frequency deviates from a rhythmic condition.

27. Method according to claim 26, wherein a monotonous correction function σ is used depending on said correction parameter α and assuming values from 0 to 1, wherein said correction function σ assumes the value of 0 if said correction parameter α equals 1 and said correction function σ assumes the value of 1 for said correction parameter α tending to infinity.

28. Method according to claim 27, wherein said effective amplitude A_{eff} is provided as a quotient, the dividend of said quotient comprising the sum of said variance and the product of said correction function σ and the square of the mean value and the divisor of said quotient comprising the mean value.

29. Method according to claim 27, wherein said effective amplitude d_{eff} is provided as a quotient with a dividend comprising the spectral density S(ω) at ω=0 and a divisor comprising the sum of said variance and the product of said correction function σ and the square of the mean value.

30. Method according to claim 20, wherein said spectral density S(ω) is determined as the Fourier Transformation of the autocorrelation of said curve.

31. Method according to claim 20, wherein the spectral density S(ω) at ω=0 is provided as said variance multiplied by a constant factor.

32. Method according to claim 20, wherein a comparative value of at least one of said physiological parameters is determined using pulse contour algorithms.

33. Method according to claim 20, further comprising
inputting information about a traveling deviation of an intrinsic physical quantity administered to the blood stream of said patient at a first location of the blood circulation of said patient, and
reading in measurement readings of said physical quantity at a second location of the blood circulation of said patient over the course of time,
wherein, said measurement readings of said physical quantity at said second location of the blood circulation of said patient are recorded as a dilution curve, and said comparative value of at least one of said physiological parameters is determined from said dilution curve using dilution algorithms.

34. Method according to claim 32, wherein said comparative value is used for calibration.

35. Method according to claim 34, wherein calibration includes determining, using said comparative value, correction parameter α used in determining said model parameter, said correction parameter α assuming values greater than or equal to 1, said values being the higher the less the patient's heart frequency deviates from a rhythmic condition.

36. Method according to claim 32, wherein said physiological parameter is rejected and re-calculated, if the difference between the determined physiological parameter and the respective comparative value exceeds a threshold value.

37. Method according to claim 20, wherein said blood variable is an arterial blood pressure, said curve representing said variable over time t is a pressure curve P(t) over time t, and said mean value is a mean arterial blood pressure <P>.

38. Method according to claim 20, wherein said imported readings are readings obtained through plethysmographic and/or pulsoximetric measurement techniques.

39. Physical storage medium having stored thereon a computer program which, when executed on a computer system, causes the computer system to perform a method for determining at least one physiological parameter of a patient, said method comprising the steps of:
importing readings of a blood variable of said patient,
storing said readings as a curve representing said blood variable over time t,
determining a mean value from said curve and determining at least one physiological parameter using said mean value
wherein
said method further includes
determining at least one of a spectral density S(ω) of said curve and a variance of said blood variable,
determining at least one model parameter representing an effective value of a heart beat using said mean value and at least one of said spectral density S(ω) and said variance <(δP)²>, said effective value being selected from an effective amplitude A_{eff} of said heart beat, an effective duration d_{eff} of said heart beat and an effective area F_{eff} under said heart beat, and
determining said at least one physiological parameter using at least one said model parameter.
